(19) United States
(12) Patent Application Publication
(10) Pub. No.: US 2024/0374405 A1
(43) Pub. Date: Nov. 14, 2024

(54) Stent With Enhanced Deployment Characteristics

(71) Applicant: MicroVention, Inc., Aliso Viejo, CA (US)

(72) Inventor: Hussain Rangwala, Villa Park, CA (US)

(73) Assignee: MicroVention, Inc., Aliso Viejo, CA (US)

(21) Appl. No.: 18/292,777

(22) PCT Filed: Jul. 29, 2022
(86) PCT No.: PCT/US2022/074299

§ 371 Date: Jan. 26, 2024

Related U.S. Application Data
(60) Provisional application No. 63/227,946, filed on Jul. 30, 2021.

(51) Int. Cl. A61F 2/915

(52) U.S. Cl. CPC A61F 2/915; A61F 2002/91575; A61F 2002/91591; A61F 2220/0033; A61F 2250/0029

(57) ABSTRACT

A stent with enhanced deployment characteristics to prevent twisting of the stent during delivery or deployment. The stent may include a first wire segment and a second wire segment that are linked together by at least one connector. The first wire segment may be rotatably connected to a first side of the at least one connector and the second wire segment may be rotatably connected to a second side of the at least one connector. The at least one connector may include an elongated body having at least two passages including a first passage for receiving the first wire segment and a second passage for receiving the second wire segment. The first and/or second wire segments may include enlargements to prevent the wire segments from being pulled out of the passages.

### RELATED APPLICATIONS

This application claims priority to U.S. Provisional Application Ser. No. 63/227,946 filed Jul. 30, 2021 entitled Stent with Enhanced Deployment Characteristics, which is hereby incorporated herein by reference in its entirety.

### BACKGROUND OF THE INVENTION

Stents are used in a number of different medical applications advantageously where they are deployed within a patient's vessel. While some vessel regions are relatively straight, many vessel regions have some type of curvature associated with their structure, which can range from relatively shallow curves to highly tortuous curves that bend in one or more directions.

Generally, most stents are easiest to deploy within relatively straight or gently curving regions of a vessel. These regions allow the stent to radially expand uniformly throughout its length to desirably anchor against the inner vessel surface. However, highly tortuous regions of vessel can cause a stent to bend and twist in several different direction. This bending and twisting can be particularly challenging for stents braided from one or more wires, since the bending and twisting of the braided wires against each other can cause pressure and friction against each other, which then can prevent movement relative to each other. In turn, this lack of movement of the braided wires relative to each other can result in a failure of the stent to fully radially expand and therefore anchor within the patient's vessel.

Hence, there is a need to improve stent deployment such that stents can be more uniformly and consistently deployed in highly tortuous regions of a vessel.

### SUMMARY OF THE INVENTION

Disclosed herein is a stent configured to radially expand and deploy more uniformly within a tortuous region of a vessel. This improved expansion and deployment can be achieved, in part, by reducing or eliminating twisting of individual wires of a braided stent that can occur when being deployed in a particularly tortuous vessel region.

In one embodiment, one or more anti-twist connectors are used to reduce or eliminate twisting of the stent and/or its braided wires. More specifically, a stent may include one or more anti-twist connectors that connect at least two segments of wire together longitudinally and allow those segments to rotate or turn relative to each other.

In one embodiment, the anti-twist connector comprises a body having two adjacent passages extending partially or completely therethrough that are each sized to accommodate the diameter of one of the braided stent wires that make up the stent. A distally extending stent wire segment passes into and is engaged with one passage and a proximally extending stent wire segment passes into and is engagement with the second passage. Both wire segments can be engaged with the passages in a manner that allows the segments to rotate relative to each other. Therefore, a wire that would otherwise extend uninterrupted between the proximal and distal end of the stent is instead replaced by at least two wire segments connected by the anti-twist connector to allow rotation relative to each other.

A stent may include a single anti-twist connector or may include a plurality of anti-twist connectors. Such a plurality of anti-twist connectors can all be located at the same longitudinal location along the length of the stent (e.g., about a quarter, half, or two thirds the length of the stent) or the plurality of anti-twist connectors can be located at several different locations along the length of the stent (e.g., about a quarter, half, and/or two thirds the length of the stent).

As a stent with one or more anti-twist connectors is deployed in a tortuous vessel, its body and constituent wires may begin to twist and therefore resist uniform radial expansion. However, the one or more anti-twist connectors release this twisting tension by allowing their connected wire segments to rotate relative to each other. Hence, the stent is able to expand in a more uniform and reliable manner.

A stent may include a first wire segment, a second wire segment, and at least one connector for linking the first wire segment with the second wire segment. The first wire segment may be connected to a first side of the at least one connector and the second wire segment may be connected to a second side of the at least one connector so as to reduce twisting between the first wire segment and the second wire segment when the stent is deployed in or delivered through a tortuous vessel.

In some example embodiments, the first wire segment may be rotatably connected to the first side of the at least one connector and the second wire segment may be rotatably connected to the second side of the at least one connector.

In some example embodiments, the first side of the at least one connector may include a first opening for receiving the first wire segment and the second side of the at least one connector may include a second opening for receiving the second wire segment.

In some example embodiments, the first side or the second side of the at least one connector may include a third opening for receiving the first wire segment or the second wire segment.

In some example embodiments, the at least one connector may include a first passage for receiving the first wire segment and a second passage for receiving the second wire segment.

In some example embodiments, the first wire segment may extend fully through the first passage and the second wire segment may extend fully through the second passage.

In some example embodiments, the first wire segment may be rotatable within the first passage and the second wire segment may be rotatable within the second passage.

In some example embodiments, the first wire segment and/or the second wire segment may include a lubricous coating for reducing friction.

In some example embodiments, a distal end of the first wire segment may include a first enlargement and a distal end of the second wire segment may include a second enlargement, the first and second enlargements functioning as stoppers.

In some example embodiments, the first enlargement may comprise a first collar attached to the first wire segment and the second enlargement may comprise a second collar attached to the second wire segment.

In some example embodiments, the first wire segment may include a third enlargement proximally spaced away from the first enlargement and the second wire segment may include a fourth enlargement proximally spaced away from the second enlargement.

In some example embodiments, the first and fourth enlargements may be positioned on a second side of the at least one connector and the second and third enlargements may be positioned on a first side of the at least one connector.

In some example embodiments, the stent may comprise a laser cut stent.

In some example embodiments, the stent may comprise a braided stent.

In some example embodiments, the at least one connector may include a first hypotube and a second hypotube, the first and second hypotubes each extending between a first side and a second side of the at least one connector.

In some example embodiments, the first wire segment may be rotatably connected within the first hypotube and the second wire segment may be rotatably connected within the second hypotube.

A connector for linking a first wire segment and a second wire segment of a stent may comprise an elongated body including a first side and a second side, a first passage on the first side of the elongated body for receiving the first wire segment of the stent, and a second passage on the second side of the elongated body for receiving the second wire segment of the stent.

In some example embodiments, a first bearing may be connected within the first passage for receiving the first wire segment of the stent and a second bearing may be connected within the second passage for receiving the second wire segment of the stent.

In some example embodiments, the connector may include a third passage on the first side or the second side of the elongated body.

In some example embodiments, the first and second passages may each extend only partially through the elongated body.

A stent may comprise a fire wire segment, a second wire segment, and at least one connector means for reducing twisting between the first and second wire segments. The first wire segment may be rotatably connected to a first side of the at least one connector means and the second wire segment may be rotatably connected to the second side of the at least one connector.

In some example embodiments, the at least one connector means may be comprised of an elongated body including a first passage for receiving the first wire segment and a second passage for receiving the second wire passage.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1A is a side view of a stent with one or more anti-twist connectors according to one embodiment.

FIG. 1B is a side view of the stent of FIG. 1A in a curved position.

FIG. 2 is a side view of a stent with one or more anti-twist connectors according to one embodiment.

FIG. 3 is a side view of a stent with one or more anti-twist connectors according to one embodiment.

FIG. 4 is a side view of a stent with one or more anti-twist connectors according to one embodiment.

FIG. 5 is a perspective view of an anti-twist connector according to one embodiment.

FIG. 6 is a perspective view of an anti-twist connector linking two wire segments of a stent according to one embodiment.

FIG. 7 is a perspective view of an anti-twist connector linking two wire segments of a stent according to one embodiment.

FIG. 8 is a perspective view of an anti-twist connector linking two wire segments of a stent according to one embodiment.

FIG. 9 is a perspective view of an anti-twist connector linking multiple wire segments of a stent according to one embodiment.

FIG. 10 is a perspective view of an anti-twist connector linking multiple wire segments of a stent according to one embodiment.

FIG. 11 is a perspective view of an anti-twist connector linking multiple wire segments of a stent according to one embodiment.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements. While different embodiments are described, features of each embodiment can be used interchangeably with other described embodiments. In other words, any of the features of each of the embodiments can be mixed and matched with each other, and embodiments should not necessarily be rigidly interpreted to only include the features shown or described.

Disclosed herein is a stent configured to radially expand and deploy more uniformly within a tortuous region of a vessel. This improved expansion and deployment can be achieved, in part, by reducing or eliminating twisting of individual wires of a braided stent that can occur when being deployed in a particularly tortuous vessel region.

Each of the example embodiments shown and described herein is merely an example of a configuration for reducing or eliminating twisting of an elongated, tubular medical device such as a stent and/or its braided/laser cut wires. It should be appreciated that such example embodiments are not meant to be limiting in scope.

Although stents are primarily discussed within this specification and shown in the drawings, it should be appreciated that the systems and methods shown and/or described herein may also be utilized in connection with various other elongated, tubular medical devices which are delivered through a vasculature. Thus, it should be appreciated that the term “stent”, as used herein, may be construed as encompassing a wide range of elongated, tubular medical devices such as flow diverters and the like.

Although stents may be discussed herein as specifically comprising braided or laser cut stents, it should be appreciated that the systems and methods shown and described herein may be equally applicable to both braided and laser cut stents, as well as other configurations of stents known in the art. Thus, any reference to a “braided stent” or a “laser cut stent” should not be construed as limited to any particular type of stent, but instead should be construed as encompassing any and all types of stents known in the art, including stents that may not be braided or laser cut.

By way of non-limiting example, the systems and methods shown and/or described herein may be utilized in connection with any of the stents shown and/or described in U.S. Pat. Nos. 10,898,203, 10,617,544, 10,543,113, 10,463,515, 10,039,655, 9,867,725, 9,439,791, 8,998,679, 8,872,062, 8,562,667 and U.S. Patent Publication Nos. 2020/0138609 and 2006/0287706, all of which are hereby incorporated by reference.

Various elongated, tubular medical devices such as stents, flow diverters, and the like may have a propensity to get twisted when traversing tortuous vasculatures such as but not limited to the carotid artery. Such a propensity to twist may be particularly pronounced in longer stents. When twisted during delivery, it may become difficult to open (e.g., expand) such medical devices. This may result in an operator needing to withdraw or retract the medical device from the body and start again which, in a medical emergency or situation where time is of the essence, may result in additional time under anesthesia, cost, complications, or adverse patient outcomes. The use of anti-twist connectors may aid in reducing or eliminating the likelihood that such stents will twist when being deployed or delivered to a target location in a patient.

The present invention is generally directed to a stent having two or more segments that may be interconnected with each other via one or more anti-twist connectors. The two or more segments of the stent may be substantially similar (e.g., have the same or similar braiding, laser cut patterns, lengths, widths, and/or other characteristics) or may be different from each other (e.g., have different braiding, laser cut patterns, lengths, widths, and/or other characteristics).

Generally, twisting may occur throughout the length of the stent, such as but not limited to the approximate center of the length of the stent. Thus, the present invention may utilize one or more anti-twist connectors linking together a pair of segments of a stent that are equal in length. However, it should be appreciated that various other positions along the length of the stent may be utilized for the anti-twist connector. For example, it may be desirable to position the at least one anti-twist connector closer to the distal or proximal end of the stent. As a further example, in longer stents, it may be desirable to split the stent into three or more segments, with each of the segments being interconnected by one or more anti-twist connectors.

The number of anti-twist connectors may vary depending on the characteristics of the stent, including its length, width, type, configuration, and the like. For example, a single anti-twist connector may be utilized. However, additional anti-twist connectors may be utilized to improve the function of the present invention. Such anti-twist connectors may be positioned radially around the circumference of the stent. The anti-twist connectors may be aligned radially about the stent or may be offset with respect to each other.

The anti-twist connector may comprise an elongated body having a first side (e.g., a proximal side) and a second side (e.g., a distal side). The elongated body may have a generally curved outer profile so as to avoid any sharp edges or corners. One or more wires of a first segment of a stent may be connected to the first side of the anti-twist connector and one or more wires of a second segment of the stent may be connected to the second side of the anti-twist connector.

The anti-twist connector may include one or more passages on each of its sides. For example, the anti-twist connector may include a single passage on its first side for receiving a wire from the first segment of the stent and a single passage on its second side for receiving a wire from the second segment of the stent. However, the number of passages on either side of the anti-twist connector may vary. As an example, a first side of the anti-twist connector may have a single passage for receiving a single wire from the first segment of the stent and a second side of the anti-twist connector may have a pair of passages for receiving a pair of wires from the second segment of the stent, or vice versa. The present invention may also utilize three or more passages on one or both sides of the anti-twist connector to allow for even more wires from one or both segments of a stent to be connected thereto.

The manner by which the wires of the stent are connected to the anti-twist connector may vary. The wires may be fixed within the anti-twist connector, may pass through the anti-twist connector, or may be rotatably connected within the anti-twist connector. The anti-twist connector may include passages comprised of openings through which a wire may extend fully or partially (e.g., the wire may extend completely through the anti-twist connector or may terminate therein). The anti-twist connector may include various tubular devices such as a bearing into which the wire may be inserted. The wire may rotate with respect to the anti-twist connector, or the wire may be fixed to such a tubular device, with the tubular device itself rotating with respect to the anti-twist connector.

One or more enlargements may be connected to or formed on the wire to prevent the wire from advancing too far through the anti-twist connector or withdrawn and becoming disconnected from the anti-twist connector. The one or more enlargements may comprise a separate structure, such as a bead or the like, which is fixed to the wire. As another example, the one or more enlargements may comprise a ball of adhesive, metal, alloys, plastic, or the like. Each wire may include an enlargement at its terminal distal end. Additionally or alternatively, each wire may include an enlargement which is inset (e.g., spaced away from) from its terminal distal end.

Portions of the wire may be coated with various coatings to reduce friction of the wire as it rotates within the anti-twist connector. Various coatings such as lubricous coatings known in the art may be utilized. For example, the terminal end of a wire may be coated with a lubricous coating before being inserted into the passage of the anti-twist connector so as to reduce friction as the wire rotates within the passage. Alternatively or additionally, such a lubricous coating may be introduced into the passage of the anti-twist connector prior to inserting the wire therein. The coating may comprise a layer of a polymeric or fibrous material such as PTFE or Teflon that is positioned around the wire and/or within the inner diameter of the passage to reduce friction.

Specific example embodiments are described further below. However, it should be understood that any of the features from any of the embodiments can be mixed and matched with each other in any combination. Hence, the present invention should not be restricted to only these embodiments, but any broader combination thereof.

In one embodiment, one or more anti-twist connectors are used to reduce or eliminate twisting of the stent and/or its braided wires. More specifically, a stent may include one or more anti-twist connectors 100 that connect at least two discrete segments of wire together to form a single elongated wire strand and allow those segments to rotate or turn relative to each other.

Generally, each of the anti-twist connectors 100 connect to a terminal end of a proximally extending wire segment and to another terminal end of a distally extending wire segment. In this respect, an anti-twist connector 100 connects two wire segments into a single, unitary wire. The proximally extending and distally extending wire segments are preferably connected to the anti-twist connector 100 in a manner that allows them to rotate relative to each other. Hence, as portions of the stent twist relative to each other during deployment in a tortuous vessel, the wire segments may rotate relative to each other to reduce tension caused by the twisting and thereby allow for more uniform radial expansion of the stent.

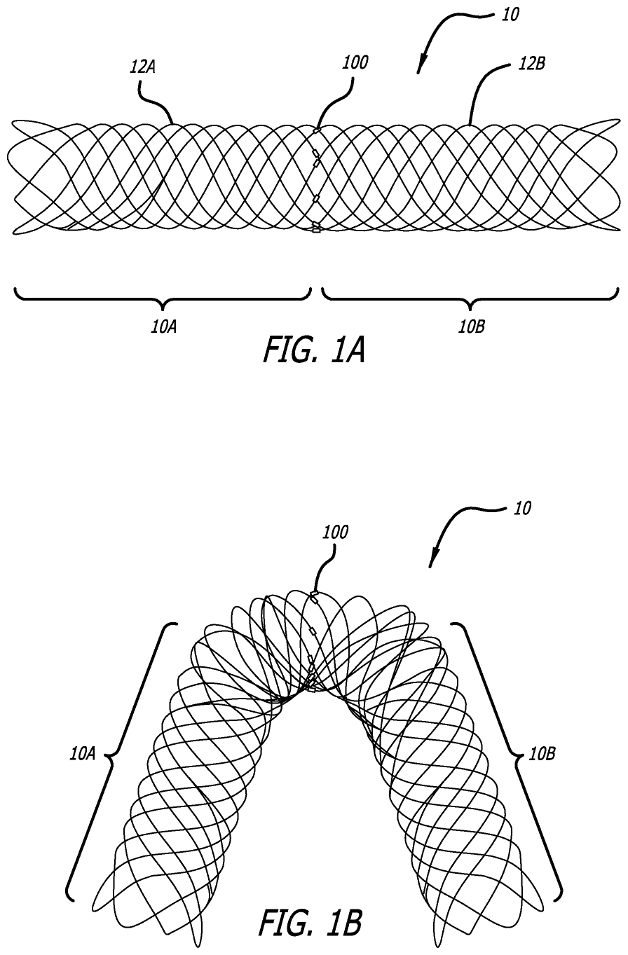

FIGS. 1A-3 illustrate example embodiments of a stent 10; each having a plurality of anti-twist connectors 100 linking a proximal region 10A including a proximal wire segment 12A with a distal region 10B including a distal wire segment 12B. FIGS. 1A-1B illustrate a first example embodiment of a braided stent 10 having uniform braiding across both regions 10A, 10B and wire segments 12A, 12B.

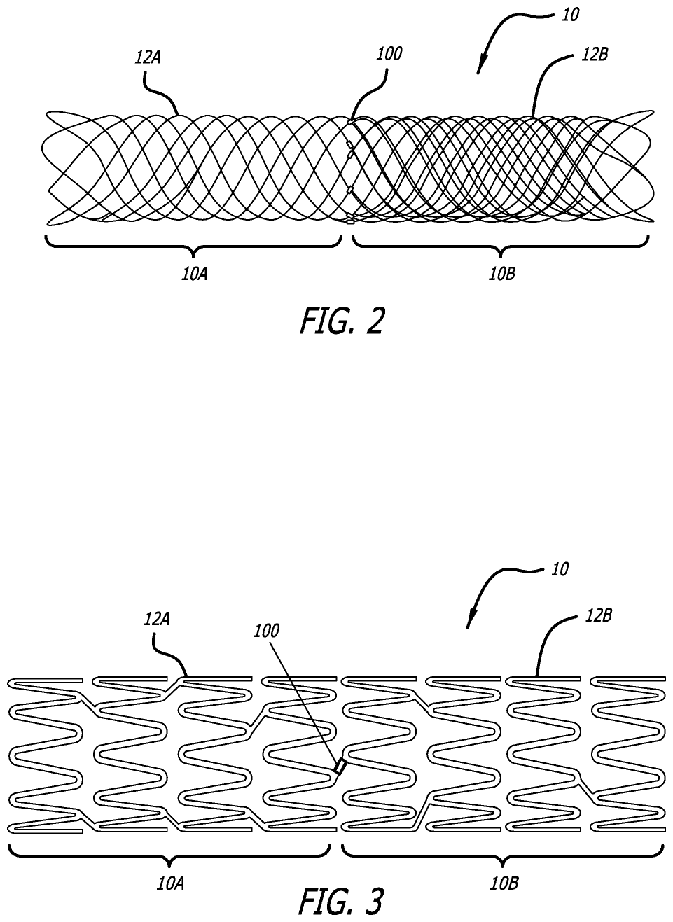

FIG. 2 illustrates an example embodiment of a braided stent 10 having different braiding across both regions 10A, 10B and wire segments 12A, 12B. More specifically, it can be seen in FIG. 2 that the distal region 10B including the distal wire segment 12B may include more braiding than the proximal region 10A including the proximal wire segment 12A. It should be appreciated that the reverse configuration may also be utilized in some example embodiments (e.g., the proximal region 10A including the proximal wire segment 12A may include more braiding than the distal region 10B including the distal wire segment 12B).

FIG. 3 illustrates an example embodiment of a laser-cut stent 10 including at least one anti-twist connector 100 linking a proximal region 10A with a distal region 10B. While FIG. 3 illustrates an embodiment in which the proximal and distal regions 10A, 10B are substantially similar, it should be appreciated that the proximal and distal regions 10A, 10B may differ in various respects in different embodiments.

FIGS. 1A-3 each illustrate example embodiments in which one or more anti-twist connectors 100 are positioned at the approximal middle of the length of the stent 10. Thus, in such embodiments, the length of the proximal region 10A may be approximately equal to the length of the distal region 10B. However, in some embodiments, the one or more anti-twist connectors 100 may not be positioned at the middle of the length of the stent 10. For example, the anti-twist connectors 100 may be connected closer to the proximal end or the distal end of the length of the stent 10 in some example embodiments.

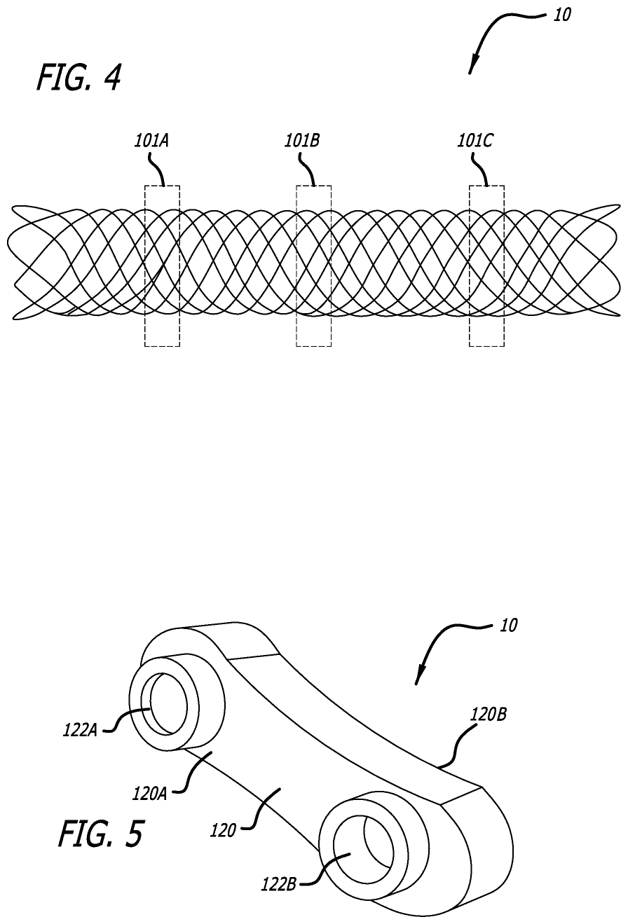

While FIGS. 1A-3 illustrate example embodiments in which only two separate regions of the stent 10 are linked by anti-twist connectors 100, it should be appreciated that three or more regions of the stent 10 may be linked together in some embodiments. For example, FIG. 4 illustrates an example embodiment in which it can be seen that multiple anti-twist connectors 101A, 101B, 101C may be connected at various locations along the length of an example embodiment of a stent 10. Thus, in the embodiment shown in FIG. 4, the stent 10 may be separated into four regions: a first region linked to a second region by anti-twist connectors 101A, the second region linked to a third region by anti-twist connectors 101B, and the third region linked to a fourth region by anti-twist connectors 101C.

It should be appreciated that various other configurations not shown in FIGS. 1A-4 may be supported by the methods and systems described herein. For example, in an example embodiment, three regions of a stent 10 may be linked by anti-twist connectors 100. In other example embodiments, five or more regions of a stent 10 may be linked by anti-twist connectors 100.

It should also be appreciated that the respective lengths of each region of a stent 10 separated by anti-twist connectors 100 may vary in different embodiments. The figures illustrate various embodiments in which the respective lengths of each region 10A, 10B are approximately equal. For example, FIGS. 1A-3 illustrate embodiments in which a first region 10A may be approximately equal in length to a second region 10B. FIG. 4 illustrates an embodiment in which the stent 10 is separated into four regions having approximately equal lengths. However, in some embodiments, one or more regions of the stent 10 may be longer than others.

Thus, the anti-twist connectors 100 need not necessarily be equally spaced such as shown in the figures. For example, with respect to the embodiment shown in FIG. 1A, the first region 10A may in some embodiments be longer than the second region 10B, or vice versa. As a further example, with respect to the embodiment shown in FIG. 4, one or more regions may be longer than one or more other regions such that the anti-twist connectors 100 are not equally spaced as shown.

The number, positioning, and orientation of anti-twist connectors 100 linking each region of the stent 10 may also vary in different embodiments. In the example embodiments shown in the figures, it can be seen that the anti-twist connectors 100 may be substantially aligned along a vertical axis bisecting the stent 10. However, the anti-twist connectors 100 may in some embodiments not be vertically aligned as shown in the figures. Additionally, the number of anti-twist connectors 100 linking each region of a stent 10 may vary in different embodiments and thus should not be construed as limited by the exemplary figures. More or less anti-twist connectors 100 may be utilized than are shown in the figures depending on various characteristics (e.g., width, materials) of the stent 10, the application for which the stent 10 is being used, the vasculatures through which the stent 10 is traversed, and/or other considerations.

FIGS. 1A and 1B illustrate one example stent 10 that has a plurality of anti-twist connectors 100 connected to a proximal wire segment 12A from region 10A and to a distal wire segment 12B from region 10B. As seen in FIG. 2, when the stent 10 is deployed in a tortuous vessel, one portion of the stent 10 may twist relative to another portion (e.g., proximal region 10A relative to distal region 10B). However, the anti-twist connectors 100, here located near the middle of the stent 10, allow one or more wire segments in the proximal region 10A to rotate relative to one or more wire segments in the distal region 10B, so as to thereby relieve tension or friction caused by the twisting that might otherwise prevent the stent 10 from radially expanding in a desired manner.

In one embodiment, the stent 10 is formed from a plurality of discrete wires (e.g., 64) that are braided together in a tubular shape. In another embodiment, the stent is initially braided from a single wire to form a tubular shape and then connectors 100 are later added at desired locations. This may involve cutting the initially single wire, after it has been braided, at the desired location into two sub-segments that are then re-attached to each other via a connector 100. In yet another embodiment, the stent can be a laser cut stent with a plurality of struts such as shown in FIG. 3. In any of these embodiments, wire or strut segments represent portions of wire or strut that extend only partially between the proximal and distal ends of the stent 10. Hence, multiple wires/strut segments may be connected to fully extend between the proximal and distal end of the stent 10.

FIG. 1A shows that the proximal and distal wire segments 12A, 12B connected with the anti-twist connector 100 may be about equal lengths (i.e., the connector 100 may be located at about the middle of the stent 10). However, one or more connectors 100 can be located at one or more locations along the length of the stent 10, as seen in FIG. 4. For example, the one or more connectors can be located at about 25% (101A), 50% (101B), and/or 75% (101C) of the length of the stent 10. In that respect, the length of an entire strand of wire between the proximal and distal ends of the stent 10 may be broken into 2, 3, 4 or more wire segments that are all connected by the anti-twist connectors 100.

In addition to the connectors 100 being located at different positions along the length of the stent 10, the connectors can be used to connect all wires at a specific cross-sectional location (e.g., all wires that intersect a middle/50% location on the stent 10) or can connect with less than all of wires at a specific cross-sectional location. For example, when viewing a cross section of the stent 10 at a given location and moving clockwise, a connector may be used with every other wire, every third wire, every fourth wire, every fifth wire, or a similar pattern (i.e., wire here meaning two connected wire segments). Described another way, the connectors may be positioned only on wires at specific circumferential angles when viewing a cross section of the stent 10, such as 0 degrees and 180 degrees.

In another example, each wire (i.e., extending between the proximal and distal ends of the stent 10) may have one or more connectors 100 that alternate in their longitudinal position along the stent 10. For example, from a cross sectional, clockwise perspective, a first connector may be located on a first wire at 25% of the stent length, a second connector may be located on a second adjacent wire at 75% of the stent length, a third connector may be located on a third adjacent wire at 25% of the stent length, and so on in that pattern. The order of connections between one wire segment to the strands of the other segment with the anti-twist connector 100 in a braided stent may depend on the level of torquing freedom needed when deploying the stent in a long and/or tortuous blood vessels of a patient (i.e., how much twisting the stent 10 must accommodate).

In another example, the longitudinal position of the connectors 100 along the stent's length can also be varied in combination with the cross sectional or circumferential angular position of the connectors 100. For example, four connectors 100 may be located at different positions along the length of the stent 10; with each being offset from each other by 90 degrees. Such connector arrangements may impact how the anti-twist aspect of the stent 10 functions and in this way, allow the stent 10 to be calibrated to achieve specific performance characteristics.

The anti-twist connector 100 may have a number of different embodiments that primarily allow two connected wire segments rotate relative to each other. Also, the connector 100 may in a preferred embodiment be connected to the wire segments in a manner that prevents longitudinal or translational movement of the connector and/or wire segments relative to each other.

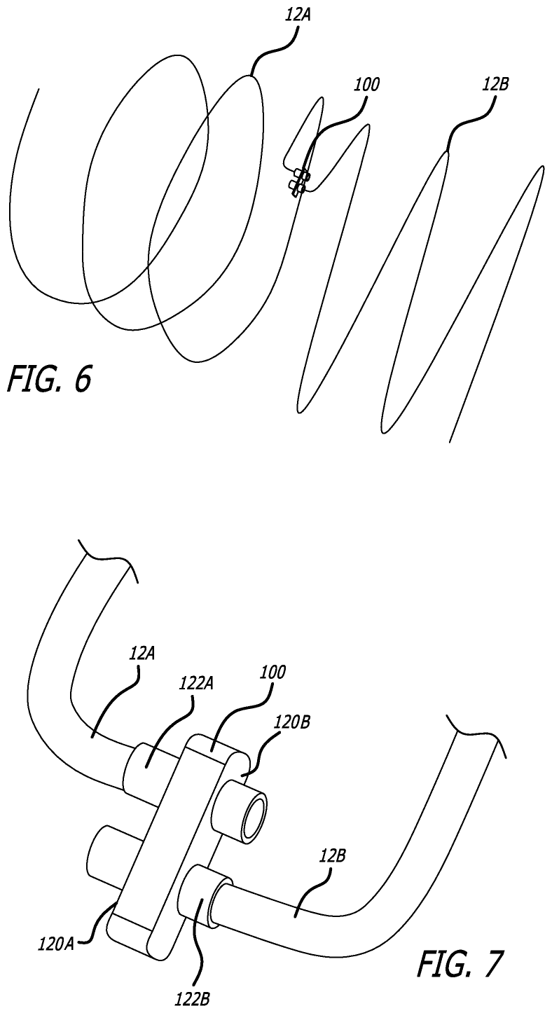

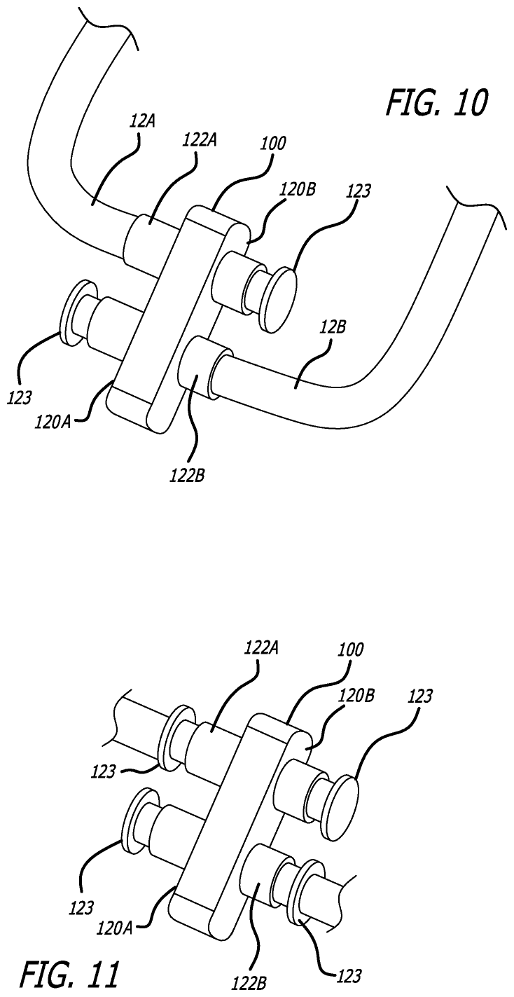

FIGS. 5-11 illustrate various example embodiments of an anti-twist connector 100 which may be utilized to link two or more regions 10A, 10B of a stent 10 so as to prevent twisting of the stent 10 during delivery or deployment. As shown throughout the figures, the anti-twist connector 100 may comprise an elongated body 120 having a first side 120A and a second side 120B. Generally, one or more first wire segments 12A may be connected to the first side 120A of the anti-twist connector 100 and one or more second wire segments 12B may be connected to the second side 120B of the anti-twist connector 100.

The shape of the anti-twist connector 100, including the shape of the elongated body 120 of the anti-twist connector 100, may vary in different embodiments and thus should not be construed as limited in scope by the example embodiments shown in the figures. In some embodiments, the elongated body 120 may be substantially rectangular. Generally, the elongated body 120 will have curved edges such as shown in the figures so as to avoid any sharp points that could injure the patient. However, in some embodiments, the elongated body 120 may include one or more corners.

FIG. 5 illustrates a first side 120A of an example embodiment of an anti-twist connector 100. As shown, the first side 120A of the elongated body 120 of the anti-twist connector 100 may include a pair of wire passages 122A, 122B each being configured to receive a wire segment 12A, 12B. As discussed in more detail below, each of the wire passages 122A, 122B may pass fully through the elongated body 120 (e.g., passing from the first side 120A to the second side 120B) of the anti-twist connector 100 in some embodiments or, in other embodiments, each of the wire passages 122A, 122B may terminate within the elongated body 120 of the anti-twist connector 100.

Generally, each passage 122A, 122B may comprise an opening extending partially or fully through the elongated body 120 of the anti-twist connector 100. The shape and dimensions of the passages 122A, 122B may vary in different embodiments and thus should not be construed as limited by the example embodiments shown in the figures.

For example, while the figures illustrate that each of the passages 122A, 122B is comprised of a circular opening, it should be appreciated that various other shapes may be utilized in different embodiments. As a further example, while the passages 122A, 122B are shown in some embodiments as passing completely through the elongated body 120 (e.g., between the first and second sides 120A, 120B of the elongated body), the passages 122A, 122B may in some embodiments terminate within the elongated body 120 and thus not pass fully therethrough.

The dimensions (e.g., the diameter) of the passages 122A, 122B may vary to suit different types and sizes of wire segments 12A, 12B. Generally, the inner diameter of each passage 122A, 122B may be slightly larger than the outer diameter of the wire segments 12A, 12B being inserted therein. However, in some embodiments, the inner diameter of each passage 122A, 122B may be greater than the outer diameter of the wire segments 12A, 12B.

The number of passages 122A, 122B of the anti-twist connector 100, and thus the number of wire segments 12A, 12B connected to the anti-twist connector 100, may vary in different embodiments and thus should not be construed as limited by the exemplary figures. FIGS. 7-8 and 10-11 illustrate example embodiments in which each side 120A, 120B of the elongated body 120 of the anti-twist connector 100 includes a pair of passages 122. FIG. 9 illustrates an example embodiment in which the first side 120A of the elongated body 120 of the anti-twist connector 100 includes a pair of first passages 122A and the second side 120B of the elongated body 120 of the anti-twist connector 100 includes a single passage 122B. The reverse configuration could be utilized in other embodiments. Additionally, one or both sides 120A, 120B of the elongated body 120 of the anti-twist connector 100 may include three or more passages 122 in some embodiments.

FIGS. 5-11 illustrate example embodiments of an anti-twist connector 100 comprising an elongated body 120 with a first wire passage 122A and a second wire passage 122B that are each sized to allow a terminal end of a wire segment 12A, 12B to be positioned therewithin. Depending on how the wire segments 12A, 12B are engaged with the connector 100, the passages 122 may extend partially through the elongated body 120 or completely through the elongated body as previously mentioned.

The elongated body 120 can take the form of a variety of different shapes and sizes, depending on the size of the stent 10 it is located on and/or the stent's wire diameter size. For example, the elongated body can form a rectangular cuboid, square cube, oval shape, spherical shape, or various other shapes. Thus, the shapes illustrated in the exemplary embodiments of the figures should not be construed as limiting in scope.

It should be appreciated that the anti-twist connector 100, including the elongated body 120 thereof, may be composed of various types of materials known in the art. As an example, the elongated body 120 of the anti-twist connector 100 may be composed of various biocompatible materials including metals, alloys, polymers, and the like. The elongated body 120 may be composed of a metal or alloy (e.g., Nitinol) or a flexible material, such as a polymer or silicone to further increase movement of the wire segments relative to each other. In some embodiments, the elongated body 120 may comprise two or more materials which are linked (e.g., fused) together. For example, the elongated body 120 may include a core composed of a first material and an outer layer (e.g., plating) composed of a second material. The rigidity of the elongated body 120 may also vary in different embodiments. Thus, the elongated body 120 may be composed of rigid, semi-rigid, flexible, and/or resilient materials.

It should also be appreciated that the relative positioning of the wire passages 122A, 122B may vary in different embodiments. The first wire passage 122A and the second wire passage 122B may be positioned generally parallel to each other such as shown in the figures or can be positioned at various non-parallel angles relative to the axis of each passage (e.g., 5, 10, 15, 20, or 25 degrees relative to each other). In some embodiments, the first wire passage 122A and the second wire passage 122B may be directly aligned with each other.

In one example, the connector can be formed by placing two hypotubes in close proximity to each other and then welding the two hypotubes together. Alternately, the hypotubes can be welded to an existing body structure. In some examples, hypotubes may not be necessary and the first wire passage 122A and the second wire passage 122B can be created directly within an elongated body structure (e.g., by drilling, laser cutting, or various other methods known to introduce an opening in a structure).

The manner by which the wire segments 12A, 12B, such as the terminal ends of the wire segments 12A, 12B, are connected to the anti-twist connector 100 may vary in different embodiments. By way of example and without limitation, the wire segments 12A, 12B may be fixed within the passages 122, may be rotatably connected within the passages 122, or may freely pass through the passages 122.

In some embodiments, a separate structure or device may be fixed or rotatably connected within the passages 122 of the anti-twist connector 100. As a non-limiting example, a tubular member such as a bearing, a tube, or the like may be fixed or rotatably connected through the anti-twist connector 100. In such embodiments, the wire segments 12A, 12B may either rotate with respect to such a tubular member, or such a tubular member may itself rotate within the passage 122 with the wire segments 12A, 12B being fixed within the tubular member so as to rotate with the tubular member. Thus, in embodiments in which such a tubular member is utilized, the wire segments 12A, 12B may alternatively be fixed to, rotatable connected to, or freely pass through such the tubular member.

FIGS. 7 and 9 illustrate example embodiments in which the wire segments 12A, 12B do not extend fully through the passages 122, but instead terminate therein. As previously mentioned, the passages 122 may comprise openings in the elongated body 120 of the anti-twist connector 100 or, in some embodiments, a separate structure or device such as but not limited to a tubular member or bearing may be fixedly or rotatably connected with the passages 122. FIG. 7 illustrates an embodiment in which the passages 122 extend between the first and second sides 120A, 120B of the elongated body 120. FIG. 9 illustrates an embodiment in which the passages 122 do not terminate either at the outer surface or within the elongated body 120 of the anti-twist connector 100.

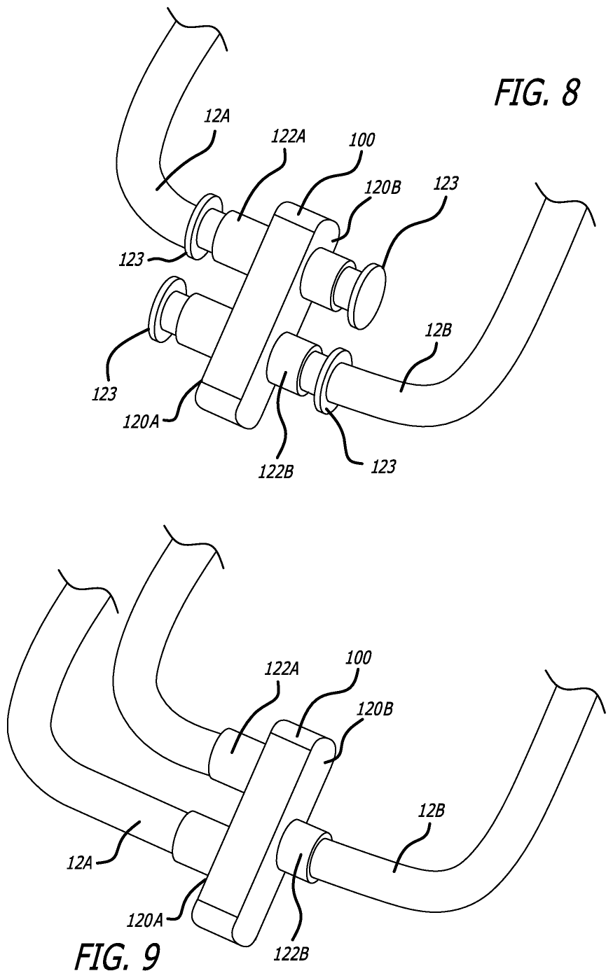

FIGS. 8 and 10-11 illustrate example embodiments in which the wire segments 12A, 12B pass fully through the elongated body 120 of the anti-twist connector 100. In such embodiments, enlargements 123 may be formed on or attached to the wire segments 12A, 12B to prevent the wire segments 12A, 12B from being withdrawn from or advanced to far through the elongated body 120 of the anti-twist connector 100 as discussed in more detail below.

As should be appreciated from FIG. 11, the orientation (e.g., angle of entry) of the wire segments 12A, 12B with respect to the anti-twist connector 100 may vary in different embodiments. In some example embodiments, the wire segments 12A, 12B may each include a 90 degree turn prior to entering to the anti-twist connector 100. In other example embodiments, the wire segments 12A, 12B may be oriented linearly in a straight line prior to entering the anti-twist connector 100. In yet other example embodiments, various other angles of entry may be utilized for one or both wire segments 12A, 12B.

In one connection example, each end of a wire segment 12A, 12B may be fixed to or within the passages 122, such as by welding, adhering with adhesives, or other methods. For example, the example embodiments shown in FIGS. 7 and 9 may utilize such a connection method. While this connection method may prevent rotation of the wire segments relative to the connector 100, the position of the elongated body 120 relative to the wire segment (e.g., perpendicular to an axis of the stent 10) provides a greater degree of movement relative of the wire segments relative to each other.

In another connection example which may be utilized with the example embodiments shown in FIGS. 7 and 9, the terminal ends of the wire segments may have enlargements 123 that are engaged by a groove or similar structure within the passages 122. Such an arrangement may allow the terminal ends of the wire segments to be retained with the passages 122 while also allowing rotation of the wire segments relative to the connector 100 and each other.

In yet another connection example which may be utilized with the example embodiments shown in FIGS. 7 and 9, a bearing (not shown) may be positioned within each passage 122A, 122B, with the terminal ends of the wire segments 12A, 12B being connected within each such bearing such that the wire segments 12A, 12B may rotated with or with respect to each bearing such that each wire segment 12A, 12B may rotate about a fixed axis or such that the anti-twist connector 100 may rotate with respect to each wire segment 12A, 12B.

In another connection example seen in FIGS. 8 and 10-11, each wire segment 12A, 12B may be positioned entirely through passages 122A, 122B, respectively; with the wire segments 12A, 12B being retained in place by enlargements 123 in the diameter of the wire segments 12A, 12B on either side 120A, 120B of the passages 122. In this respect, the wire segments 12A, 12B may be prevented from being pulled out of or further pushed through the passages 122A, 122B, thereby retaining their longitudinal or translational position relative to each other. Additionally, such a connection method may allow each of the wire segments 12A, 12B to rotate freely relative to the connector 100.

The shape, size, type, and number of enlargements 123 may vary in different embodiments and thus should not be construed as limited in scope by the exemplary embodiments shown in the figures.

The figures illustrate enlargements 123 comprised of a flat disc shape. However, various other shapes may be utilized, including square-shaped, rectangular-shaped, spherical-shaped, semi-spherical-shaped, and various other shaped enlargements 123. In some embodiments, the one or more enlargements 123 may comprise a bead having an opening through which the wire segments 12A, 12B may extend, with the bead being fixed to the wire segments 12A, 12B by adhesives, welding, or the like. In some embodiments, however, the enlargements 123 may “float” along at least a portion of the length of the wire segments 12A, 12B.

The size of the enlargements 123 may vary and should not be construed as limited by the figures. Generally, each enlargement 123 should be at least slightly larger than the opening of the passage 122 so as to prevent the enlargement 123 from entering into the passage 122 or passing therethrough.

The number of enlargements 123 utilized per wire segment 12A, 12B and/or anti-twist connector 100 may also vary and should not be construed as limited by the figures. FIGS. 8 and 11 illustrates an embodiment in which each wire segment 12A, 12B includes two enlargements 123: a first enlargement at a distal end of the wire segment 12A, 12B and a second enlargement which is set back (e.g., inset) with respect to the first enlargement. Thus, the first enlargement 123 may be positioned on the second side 120B of the anti-twist connector 100 and the second enlargement 123 may be positioned on the first side 120A of the anti-twist connector 100 in such embodiments such as shown in FIGS. 8 and 11.

FIG. 9 illustrates an embodiment in which each wire segment 12A, 12B includes only a single enlargement 123 positioned at its terminal distal end on the second side 120B of the anti-twist connector 100. Although not shown, in some embodiments each wire segment 12A, 12B may instead include only a single enlargement 123 on the first side 120A of the anti-twist connector 100.

The enlargements 123 can be formed after placing the wire segments 12A and 12B through the passages 122A and 122B. For example, a collar, tube, or C-shaped structure can be placed over the wire and welded in place. Alternatively, only additional welding may be needed. Alternatively, a spot weld can be used to enlarge an end of the wire segment, thereby forming the enlargements 123.

As seen best in FIGS. 5-11, the wire segments 12A, 12B can be curved or bent so that they extend longitudinally, parallel to an axis of the stent 10 while the length of the body 120 of the connector 100 is orientated generally perpendicularly relative to the longitudinal axis of the stent 10.

The passages 122A and 122B can be located at various distances from each other, such as but not limited to within an inclusive range of 0.001 inch and 0.05 inch. The passages 122A, 122B may be generally sized slightly larger than the wire segments 12A and 12B, such as slightly larger than a wire with a diameter within an inclusive range of about 0.0005 inch and 0.02 inch.

The connectors 100 described in this specification can be used on a wide variety of different stent 10 types and sizes. For example, the connectors 100 may provide helpful performance characteristics on neurovascular stents 10, which often have an expanded diameter within an inclusive range of about 2 mm and 10 mm, and an expanded length within an inclusive range of about 5 mm and 100 mm.

In one embodiment, a stent 10 can be created by first braiding a single or multiple wire strands to a desired stent 10 shape. A connector 100 may then be incorporated by cutting a wire of the stent 10 at a desired location along the length into a first segment 12A and a second segment 12B (e.g., at various lengths including but not limited to any of the lengths previously discussed in this specification).

The distal terminal end of at least one strand of the first stent region 10A may then be connected with a first passage 122A on a first side 120A of the connector 100 and the proximal terminal end of another strand of the second stent region 10B may be connected with a second passage 122B on a second side 120B of the connector 100. The connection to the connector 100 may be performed in any manner previously discussed (e.g., welding or creating proximal and distal enlargements on the wire segments, or various other methods). Additional stent wires may be cut and connected in a similar manner at desired locations.

In a specific example, a woven stent can be cut at 75% of the length into a first region 10A and a second region 10B. The distal ends of the one or more strands of the first region 10A may be connected with one or more connectors 100 to the proximal ends of the one or more strands of the second region 10B, thereby connecting the first stent region 10A of the stent 10 to the second stent region 10B of the stent 10 through the one or more connectors 100.

In yet another example, a woven stent can be cut at 50% of the length into a first region 10A and a second region 10B. The distal ends of the one or more strands of the first region 10A may be connected with one or more connectors 100 to the proximal ends of the one or more strands of the second region 10B. According to another example, a braided or woven stent can be cut at 25% of the length into a first region 10A and a second region 10B. The distal ends of the one or more strands of the first region 10A may be connected with one or more connectors 100 to the proximal ends of the one or more strands of the second region 10B.

Alternately, wire segments can be connected to each other via a connector prior to braiding. Hence, the stent can be braided after connecting each of the desired connectors.

In use, the stent 10 may be delivered to and deployed within a vasculature using a wide range of methods known in the art for delivering and deploying a stent 10. When the stent 10 is in a linear configuration (e.g., the stent 10 is arranged in or extended along a straight or nearly straight line) such as shown in FIG. 1A, the first wire segment 12A may be aligned with the second wire segment 12B. When the stent 10 is in a non-linear configuration (e.g., the stent 10 is bent, twisted, or curved) such as shown in FIG. 1B, the connector 100 may reduce the relative twisting between the first and second wire segments 12A, 12B as compared to any relative twisting of the stent 10. In this manner, the risk of complications (e.g., failure of the stent 10 to fully expand) during deployment within a tortuous vasculature may be reduced or eliminated.

### CLAUSES

Exemplary embodiments are set out in the following numbered clauses.

Clause 1. A method of adding a connector to a stent may comprise braiding a stent with a plurality of strands, cutting the stent along the length to form a plurality of stent segments, connecting a distal terminal end of at least one strand of a first stent segment with a first passage on the connector, and connecting a proximal terminal end of at least one strand of a second stent segment with a second passage on the connector, thereby connecting the first segment of the stent with the second segment of the stent.

Clause 2. A method according to clause 1 may comprise cutting the stent to form three stent segments, connecting a distal terminal end of at least one strand of the second connector with a first passage of a second connector, and connecting a proximal terminal end of at least one strand of a third stent segment with a second passage of the second connector.

Clause 3. A method of adding a connector to a stent may comprise braiding a stent with a plurality of strands, cutting the stent along the length to form a plurality of stent segments, connecting a distal terminal end of a first strand of a first stent segment with a first passage on a first side of the connector, connecting a distal terminal end of a second strand of the first stent segment with a second passage on the first side of the connector, and connecting a proximal terminal end of a first strand of a second stent segment with a first passage on a second side of the connector.

Clause 4. A method of adding a connector to a stent may comprise braiding a stent with a plurality of strands, cutting the stent along the length to form a plurality of stent segments, connecting a distal terminal end of a first strand of a first stent segment with a first passage on a first side of the connector, connecting a proximal terminal end of a first strand of a second stent segment with a first passage on the second side of the connector, and connecting a proximal terminal end of a second strand of the second stent segment with a second passage on the second side of the connector.

Clause 5. A method of adding a connector to a stent may comprise laser cutting a first tubular stent segment and a second tubular stent segment, connecting a distal terminal end of a first wire of the first tubular stent segment with a first passage on a first side of the connector, and connecting a proximal terminal end of a first wire of the second tubular stent segment with a first passage on a second side of the connector.

Clause 6. A method according to clause 5 may comprise connecting a distal terminal end of a second wire of the first tubular stent segment with a second passage on the first side of the connector.

Clause 7. A method according to clauses 5 or 6 may comprise connecting a proximal end of a second wire of the second tubular stent segment with a second passage on the second side of the connector.

Clause 8. A method according to any of the preceding clauses may comprise coating the terminal or distal ends of any of the strands or wires with a lubricous coating to reduce friction.

Clause 9. A method according to any of the preceding clauses may comprise coating the interior of one or more of the passages with a lubricous coating to reduce friction.

Clause 10. A method according to any of the preceding clauses may comprise inserting any of the strands or wires fully through the anti-twist connector.

Clause 11. A method according to any of the preceding clauses may comprise terminating any of the strands or wires within the anti-twist connector.

Clause 12. A method according to any of the preceding clauses may comprise connecting a first enlargement to a terminal end of any of the strands or wires to act as a stopper.

Clause 13. A method according to any of the preceding clauses may comprise connecting a second enlargement to any of the strands or wires proximally spaced away from the first enlargement.

Clause 14. A method according to any of the preceding clauses may comprise forming one or more enlargements on any of the strands or wires by forming an enlarged region such as a ball with adhesive, solder, or the like.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A stent, comprising:
a first wire segment;
a second wire segment; and
at least one connector for linking the first wire segment with the second wire segment, wherein the first wire segment is connected to the at least one connector, and wherein the second wire segment is connected to the at least one connector;
wherein the first wire segment is aligned with the second wire segment when the stent is a linear configuration; and,
wherein the at least one connector reduces the relative twisting between the first and second wire segments compared to the relative twisting of the stent when the stent is in a non-linear configuration.

2. The stent of claim 1, wherein the first wire segment and the second wire segment are each rotatably connected to the at least one connector.

3. The stent of claim 1, wherein a first side of the at least one connector includes a first opening for receiving the first wire segment and wherein a second side of the at least one connector includes a second opening for receiving the second wire segment.

4. The stent of claim 3, wherein the first side or the second side of the at least one connector includes a third opening for receiving the first wire segment or the second wire segment.

5. The stent of claim 1, wherein the at least one connector includes a first passage and a second passage, wherein the first wire segment extends fully through the first passage, and wherein the second wire segment extends fully through the second passage.

6. The stent of claim 5, wherein the first wire segment is rotatable within the first passage and wherein the second wire segment is rotatable within the second passage.

7. The stent of claim 1, wherein the at least one connector includes a lubricous coating for reducing friction caused by movement of the first wire segment and/or the second wire segment.

8. The stent of claim 1, wherein a distal end of the first wire segment includes a first enlargement and wherein a distal end of the second wire segment includes a second enlargement.

9. The stent of claim 8, wherein the first enlargement comprises a first collar attached to the first wire segment and wherein the second enlargement comprises a second collar attached to the second wire segment.

10. The stent of claim 8, wherein the first wire segment includes a third enlargement proximally spaced away from the first enlargement and wherein the second wire segment includes a fourth enlargement proximally spaced away from the second enlargement.

11. The stent of claim 10, wherein the first wire segment and the second wire segment each extend fully through the at least one connector, wherein the first enlargement and the fourth enlargement are each positioned on a second side of the at least one connector, and wherein the second enlargement and the third enlargement are each positioned on a first side of the at least one connector.

12. The stent of claim 1, wherein the stent is comprised of a laser cut stent or a braided stent.

13. The stent of claim 1, wherein the at least one connector includes a first hypotube and a second hypotube, the first hypotube and the second hypotube each extending between a first side and a second side of the at least one connector.

14. The stent of claim 1, wherein the at least one connector is positioned at an approximate mid-point along a length of the stent.

15. The stent of claim 1, wherein the at least one connector is positioned closer to a proximal end or a distal end of the stent.

16. A connector for linking a first wire segment and a second wire segment of a stent, comprising:
an elongated body including a first side and a second side;
a first opening at the first side of the elongated body, wherein the first wire segment of the stent is positioned within the first opening; and
a second opening at the second side of the elongated body, wherein the second wire segment of the stent is positioned within the second opening.

17. The connector of claim 16, further comprising a first bearing connected within the first opening and a second bearing connected within the second opening, wherein the first wire segment of the stent is positioned within the first bearing, and wherein the second wire segment of the stent is positioned within the second bearing.

18. The connector of claim 16, further comprising a third opening at the first side or the second side of the elongated body, and wherein a third wire segment of the stent is positioned within the third opening.

19. The connector of claim 16, wherein the first opening and the second opening each extend only partially through the elongated body.

20. A stent, comprising:
a first wire segment;
a second wire segment; and
at least one connector means for reducing twisting between the first wire segment and the second wire segment when the stent is in a non-linear configuration.
